(19) United States
(12) Patent Application Publication
(10) Pub. No.: US 2010/0242971 A1
(43) Pub. Date: Sep. 30, 2010

(54) RESTRAINT

(75) Inventors: Brian McGuigan, London (GB); Ryder Meggitt, Glasgow (GB)

Correspondence Address: SCHWEGMAN, LUNDBERG & WOESSNER, P.A., P.O. BOX 2938, MINNEAPOLIS, MN, 55402, US

(73) Assignee: Scottish Ambulance Service, Edinburgh (GB)

(21) Appl. No.: 12/666,207

(22) PCT Filed: Jun. 25, 2008
(86) PCT No.: PCT/GB08/02241

§ 371 Date: Jun. 8, 2010

(30) Foreign Application Priority Data
Jun. 26, 2007 (GB) GB 0712254.2

(51) Int. Cl. A61F 5/37

(52) U.S. Cl. 128/876

(57) ABSTRACT

A restraint harness for a child is described. The restraint harness comprises an upper torso restraint, a lower torso restraint and at least one attachment device for attaching the upper and lower torso restraints to a stretcher or other device. In use, the upper torso restraint and the lower torso restraint restrain independently of each other.

### FIELD OF THE INVENTION

The present invention relates to a restraint, particularly, but not exclusively, to a restraint for restraining a child on a stretcher.

### BACKGROUND TO THE INVENTION

Conventional stretchers have straps which can be passed over a user to prevent the user from rolling off the stretcher. In particular, the straps are utilised if the user is being moved on the stretcher in an ambulance.

Whilst these straps are suitable for an average sized adult, they have proven to be unsuitable for infants, children or smaller adults. Furthermore, the straps and stretcher combination are only suitable for preventing a user moving in four directions; that is side to side, vertically up and vertically down. The conventional strap system does not restrain in all six directions, that's is it does not prevent the user from sliding off the ends of the stretcher, a situation which might occur in the event of the accident.

To overcome the difficulties of moving a child, restraint harnesses have been developed. Generally these harnesses are known as “five point” harnesses as they incorporate five straps: one passing over each shoulder, one passing up each side and one passing between the legs. The five straps are secured together by, for example, a single five point buckle. The advantage of such a device is the user is restrained in all six directions.

However, the five point harness does have limitations. For example, to move a user from the prone position to the seated position requires tightening of the straps which pass over the user's shoulders. Likewise, to move the user back to the prone position requires the slackening off of the shoulder straps. Whilst the tightening and slackening steps are not difficult, they are time consuming and cause the patient discomfort. In the event of a deterioration of the patient's condition, the patient's position may have to be changed rapidly. For example, in the event of failure of the respiratory system, the patient must be rapidly moved from the seated position to the prone position to facilitate medical intervention. Additionally, moving the patient, particularly from the seated to the prone positions, without slackening off the straps can cause the harness to tighten around, and cause discomfort to, the patient's crotch and bottom.

Furthermore, conventional five point harnesses do not provide a level of adjustment which can accommodate a wide range of user sizes.

Additionally, the five point harness buckle, in use, is generally located over the user's chest. This positioning makes access to the chest awkward if required for, for example defibrillation. The positioning of the buckle also reduces the clinicians ability to observe the patient's chest to ensure it is rising and falling to the clinician's satisfaction.

It is an object of at least one embodiment of the present invention to obviate or mitigate at least one disadvantage of the prior art.

### SUMMARY OF THE INVENTION

According to a first aspect of the present invention there is provided a restraint harness for a child, the restraint harness comprising:

an upper torso restraint;

a lower torso restraint; and

at least one attachment device for attaching the upper and lower torso restraints to a stretcher or other device;

wherein, in use, the upper torso restraint and the lower torso restraint restrain independently of each other.

Providing a restraint harness in which the upper and lower torso restraints restrain independently of each other permits the angle of the users back to be adjusted without the need for adjustment of the restraint harness. This permits the user to be moved, for example, from the seated position to the prone position relatively quickly if required. Furthermore, independently restraining the upper and lower torsos means a single restraint can accommodate a greater range of sizes of user. Additionally, independent action of the upper and lower torso restraints reduces the possibility of the user suffering discomfort around their crotch or bottom when being moved between the seated and prone positions. The restraint harness is suitable for use with apparatus to which it is desired to restrain a patient for transit purposes, including but not limited to a stretcher, a gurney, a spinal board, a bed, or a spring, air or vacuum mattress.

Preferably, the restraint harness comprises members which extend in at least two directions over the user's body.

Preferably, the members are straps.

Preferably, the restraint harness comprises at least one over shoulder strap, a plurality of side straps and at least one crotch strap.

Preferably, the upper torso restraint is at least a three point restraint.

Most preferably, the upper torso restraint is a four point restraint.

Preferably, the upper torso restraint comprises an upper torso back portion, at least one shoulder strap and first and second underarm straps.

Preferably, each strap comprises a first end and a second end.

Preferably, the first end of each strap is connected to the upper torso back portion.

Preferably, the first end of each strap is fixedly connected to the upper torso back portion.

Preferably, there are first and second shoulder straps.

In one embodiment, the shoulder straps pass over the user's shoulders to the user's chest and the underarm straps pass up the user's sides to the user's chest.

Preferably, each of the underarm straps is releasably connectable to at least one of the shoulder straps or vice versa. Coupling the underarm straps to the shoulder straps maintains the chest area relatively clear for medical intervention and patient observation.

Alternatively or additionally, the underarm straps are releasably connectable to each other.

Alternatively or additionally, the shoulder straps are releasably connectable to each other.

Preferably, the shoulder straps are releasably connectable to each other by means of a neck portion.

Preferably, the neck portion comprises a neck strap.

Preferably, the upper torso back portion and shoulder straps define an aperture adapted to be passed over a user's head.

Most preferably, the back portion, shoulder straps and neck portion define an aperture adapted to be passed over a user's head. In normal use, the shoulder straps will be connected together and to fit the upper torso restraint, the shoulder straps being passed either side of the user's head whilst connected.

In some cases, it may be preferable to fit the upper torso restraint with the shoulder straps separated and attach the shoulder straps together after the straps have been passed over the user's shoulders, for example when the user is attached to one or more IV lines.

Preferably, the underarm straps releasably attach to the/each shoulder straps by means of a hook and loop fastener.

Preferably, each underarm strap defines a first portion comprising a hook fastener and a second portion comprising a loop fastener.

Preferably, the lower torso restraint is a three point restraint.

Preferably, the lower torso restraint comprises a lower torso back portion, first and second waist straps and a crotch strap.

Preferably, each strap comprises a first end and a second end.

Preferably, the first end of each strap is connected to the lower torso back portion.

Preferably, the first end of each strap is fixedly connected to the lower torso back portion.

In one embodiment, the waist straps pass over the user's hips to the user's abdomen, and the crotch strap passes between the user's legs to the user's abdomen.

Preferably, at least one of the waist straps is releasably connectable to the crotch strap.

In one embodiment, the crotch strap is adapted to be sandwiched between the waist straps.

Preferably, the waist straps are connectable to each other.

Preferably, the lower torso restraint straps are connectable to one another by means of hook and loop fasteners.

Preferably, the first waist strap comprises a securing device for securing the second waist strap and the crotch strap.

Preferably, the securing device comprises first and second elements.

Preferably, the crotch strap is adapted to be received between the first and second elements.

Preferably, the first and second elements have opposing surfaces.

Preferably, the crotch strap is adapted to be received between the opposing surfaces of the securing device elements.

Preferably, the crotch strap has an upper surface and lower surface.

Preferably, each of the upper and lower surfaces are adapted to engage with one of the opposing surfaces of the securing device elements.

Preferably, each of the upper and lower surfaces are adapted to connectably engage with one of the opposing surfaces of the securing device elements.

Preferably, the surfaces are adapted to connectably engage by a hook and loop fastener. Hook and loop fasteners are adapted to separate by a peeling action. Having the crotch strap sandwiched between the waist straps or the securing device of the first waist strap reduces the peeling effect which might arise in use.

Preferably, the upper torso restraint and the lower torso restraint are connected. Connecting the upper and lower torso restraints maintains the two components as a single unit.

Preferably, the upper torso restraint and the lower torso restraint are movably connected.

Preferably, the restraint harness further comprises at least one connection element to connect the upper torso restraint and the lower torso restraint. Having the upper and lower torso restraints connected ensures they are together when required.

Preferably, the/each connection element fixedly attaches the upper and lower torso restraints.

Preferably, the/each connection element can elastically deform. Elastic deformation permits the upper torso restraint and the lower torso restraint to move with respect to each other as, for example, the user's posture changes, whilst permitting the harness to adopt a compact shape when not in use. Elastic deformation also permits the restraint harness to be used with patients of different heights; the connection element stretching if required to fit a larger user. Elastic deformation of the connection element will also prevent the harness tightening around the patient's crotch and bottom as the patient's position is changed.

Preferably, the restraint harness is movable between a useable configuration and a stored configuration.

Preferably, the restraint harness comprises a storage device for storing the restraint harness when not in use.

Preferably, the storage device is integral with the restraint harness.

Preferably, the storage device is adapted to compress the restrain harness when in the stored configuration. Compression of the harness reduces the space occupied by the harness when not in use.

Preferably, the storage devices is elastically deformable.

Preferably, the storage device is a pocket.

Preferably, the connection element and the storage device are the same.

Preferably, the restraint harness further comprises a wash bag. A wash bag is used to contain the restraint harness during cleaning of the harness.

Preferably, the wash bag and the storage device are the same.

Preferably, there are a plurality of attachment devices.

Preferably, the at least one attachment device comprises one or more straps.

Preferably, the/each attachment devices are connected to one or both of the upper and lower torso back portions.

Preferably, the/each attachment devices are fixedly connected to one or both of the upper and lower torso back portions.

Preferably, the/each attachment devices are adapted to attach to the frame of a bed or stretcher or other similar device.

Preferably, the/each attachment devices comprises a first portion and a second portion.

Preferably, the attachment devices first portion is connected to one of the torso back portions.

Preferably, the attachment devices second portion is adapted to be connected to a frame of a bed or stretcher or other similar device.

Preferably, the attachment devices first and second portions are releasably attachable to each other.

Preferably, the attachment devices first and second portions are releasably attachable to each other by means of a buckle.

Preferably, the attachment devices second portions are adapted to connect to a number of different sizes of restraint harness.

Alternatively or additionally, one attachment device is connectable to another attachment device. Such an arrangement is useful if there is no frame for example. Two attachment devices can then be wrapped around the mattress, for example, so that the restraint harness “hugs” the mattress. This feature is particularly useful if the restraint harness is to be used with a trolley without rails on the upper tilting section.

Preferably, one attachment device is releasably connectable to another attachment device.

Preferably, one attachment device is releasably connectable to another attachment device by means of a buckle.

Preferably, the/each attachment device is adjustable in length.

Preferably, the restraint harness is adapted to permit a patient to be restrained on the user's back or the user's side. Being able to restrain the patient in the recovery position is particularly advantageous and adds to the versatility of the device. This feature is facilitated by having sufficient length adjustment in the straps.

Preferably, the upper and lower torso restrain back portions and/or the/each connection element comprise a cleanable material.

Preferably, the cleanable material is a wipe clean material.

Preferably, the upper and/or lower torso restraints comprise at least one padded portion. Padded portions will improve the comfort of the harness and help prevent pressure sores.

According to a second aspect of the present invention there is provided a restraint harness comprising;

an upper torso restraint;

a lower torso restraint; and

at least one attachment device for attaching the upper and lower torso restraints to a stretcher or other device;

wherein, in use, the upper torso restraint and the lower torso restraint restrain a patient whilst leaving the patient's chest substantially uncovered.

### BRIEF DESCRIPTION OF THE DRAWINGS

An embodiment of the present invention will now be described with reference to the accompanying drawings in which:

FIG. 1 is a perspective view of a restraint harness according to a first embodiment of the present invention;

FIG. 2 is a perspective view of the upper torso restraint of FIG. 1;

FIG. 3 is a perspective view of the lower torso restraint of FIG. 1;

FIGS. 4 to 9 are a series of perspective views of a patient being secured in the restraint harness of FIG. 1;

FIG. 10 shows a schematic view of the upper torso attachment straps and the upper torso restraint; and

FIG. 11 shows a schematic view of the lower torso attachment straps and the lower torso restraint.

### DETAILED DESCRIPTION OF THE DRAWINGS

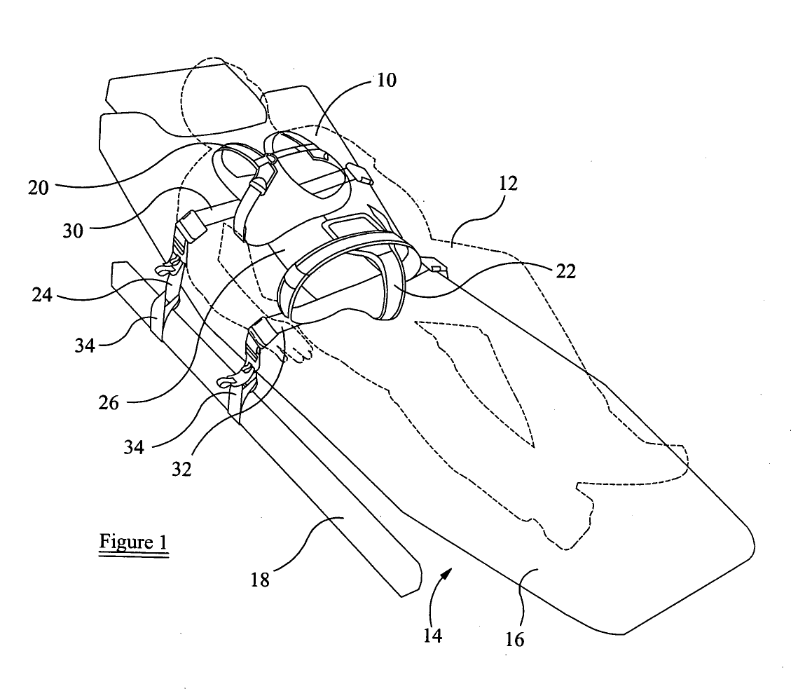

Referring firstly to FIG. 1, there is shown a perspective view of a restraint harness, generally indicated by reference numeral 10, according to a first embodiment of the present invention. The restraint harness 10 is shown restraining a patient 12 (shown in broken outline) to a stretcher 14 comprising a mattress 16 and a frame 18 (of which only part is shown for clarity).

The restraint harness 10 comprises an upper torso restraint 20, a lower torso restraint 22 and attachment devices 24. As can be seen from FIG. 1, the upper torso restraint 20 and the lower torso restraint 22 restrain the patient 12 independently of each other.

The attachment devices 24 comprise upper torso attachment straps 30 and lower torso attachment straps 32 and frame straps 34. The operation and interaction of these straps 30,32,34 will be discussed in due course.

The upper and lower torso restraints 20,22 are connected by a connection element 26 in the form of an elasticated strip of material 28. The elasticated nature of the connection element 26 permits the restraint harness 10 to be stretched to be useable with different physical sizes of patient 12.

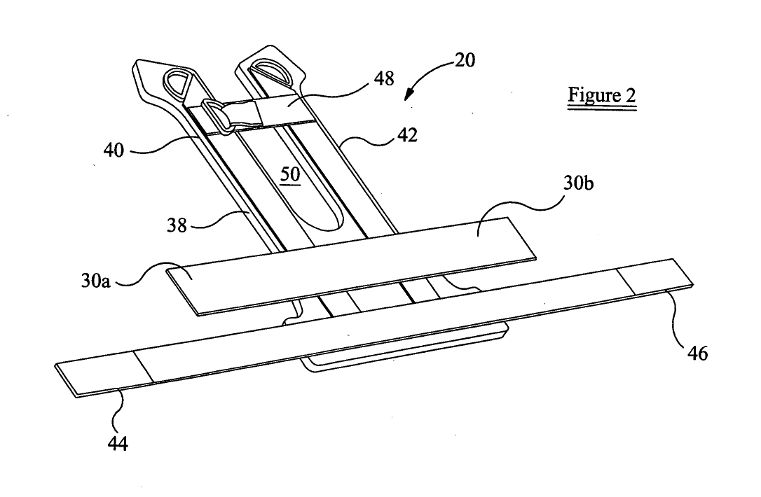

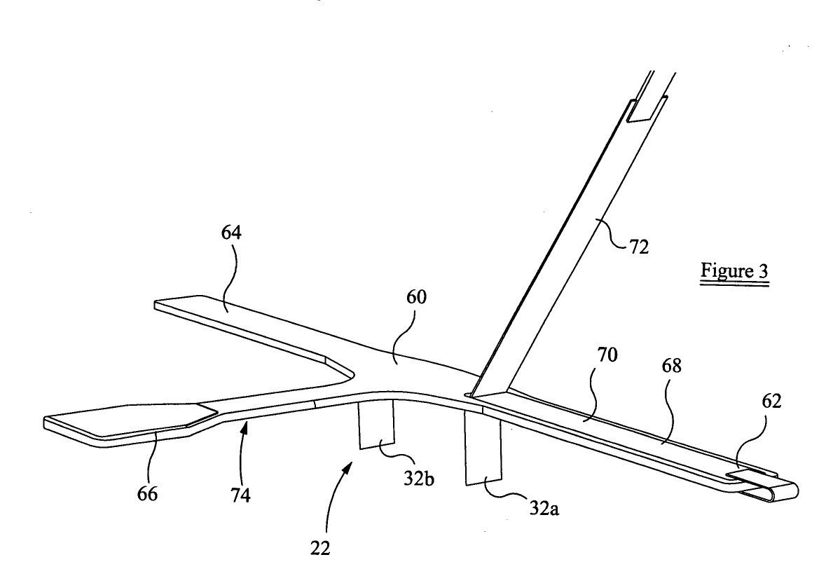

The construction of upper and lower torso restraints 20,22 will now be discussed with reference to FIGS. 2 and 3 respectively.

Referring to FIG. 2, the upper torso restraint 20 comprises an upper torso back portion 38, a first shoulder strap 40, a second shoulder strap 42, a first underarm strap 44, a second underarm strap 46. As the upper torso is restrained at four points, by the shoulder and underarm straps 40,42,44,46, the upper torso restraint 20 is a four point harness.

The upper torso restraint 20 further comprises a neck strap 48 and upper torso attachment straps 30a,30b. As can be seen from FIG. 2, the neck strap 48, the shoulder straps 40,42 and the upper torso back portion 38 define an aperture 50. The arrangement permits both shoulder straps 40,42 to be passed over a patient's head together, as will be described. The neck strap 48 is releasably attaches the shoulder straps 40,42 to each other. The neck strap 48 is stitched to the second shoulder strap 42 and passes through an eye 52 attached to the first shoulder strap 40. Once the neck strap 48 passes through the eye 52 the neck strap 48 doubles back and attaches to itself using a hook and loop fastener (not shown).

The upper torso back portion 38 comprises a padded material with a wipe clean surface 54 which stretches down the length of the shoulder straps 40,42 between, in use, the patient and the shoulder straps 40,42. This makes the restraint harness 10 more comfortable to wear and reduces the possibility of the harness 10 causing pressure sores.

Referring to FIG. 3, the lower torso restraint 22 comprises a lower torso back portion 60, a first waist strap 62, a second waist strap 64 and a crotch strap 66. As the lower torso is restrained at three points by the waist and crotch straps 62,64,66, the lower torso restraint 22 is a three point harness.

The lower torso restraint 22 further comprises attachment straps 32a,32b.

The first waist strap 62 includes a securing device 68 comprising a first element 70 and a second element 72, the purpose of which will be discussed in due course.

The lower torso back portion 60 also comprises a padded material with a wipe clean surface 74.

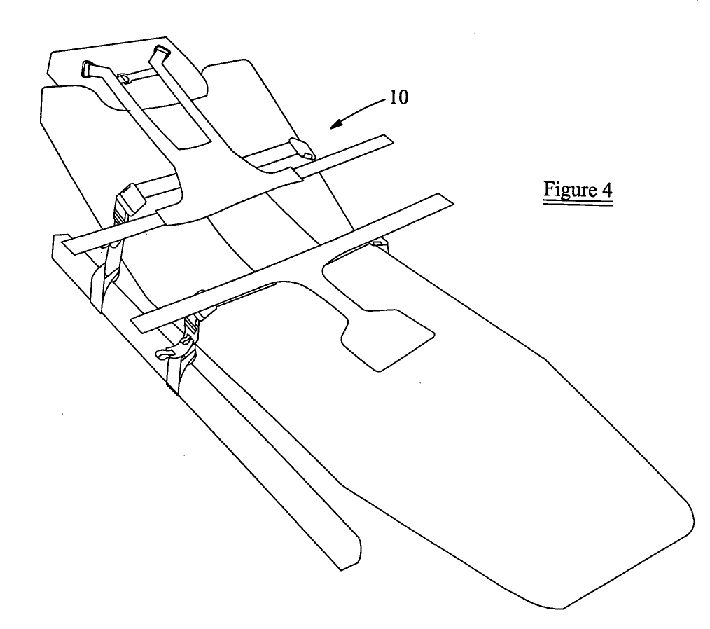

The operation of the restraint harness 10 will now be described with reference to FIGS. 4 to 9.

FIG. 4 shows the restraint harness 10 in an unfolded configuration ready to receive a patient (not shown).

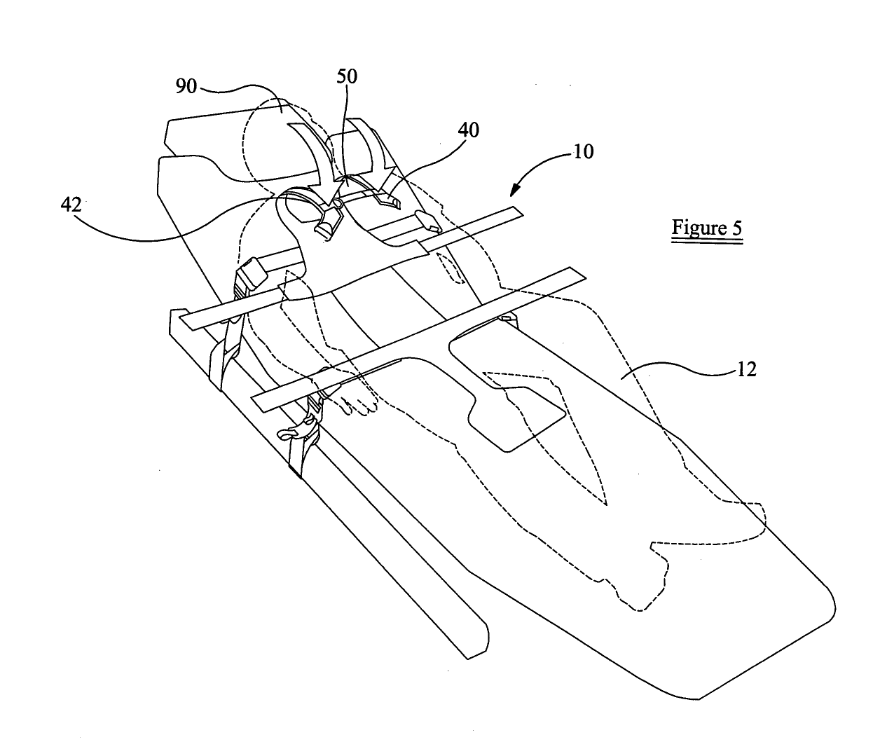

Referring to FIG. 5, the patient 12 lies on the restraint harness 10 and the stretcher 14. The shoulder straps 40,42 are passed together over the patient's shoulders in the direction of the arrows, the patient's head 90 passing through the aperture 50.

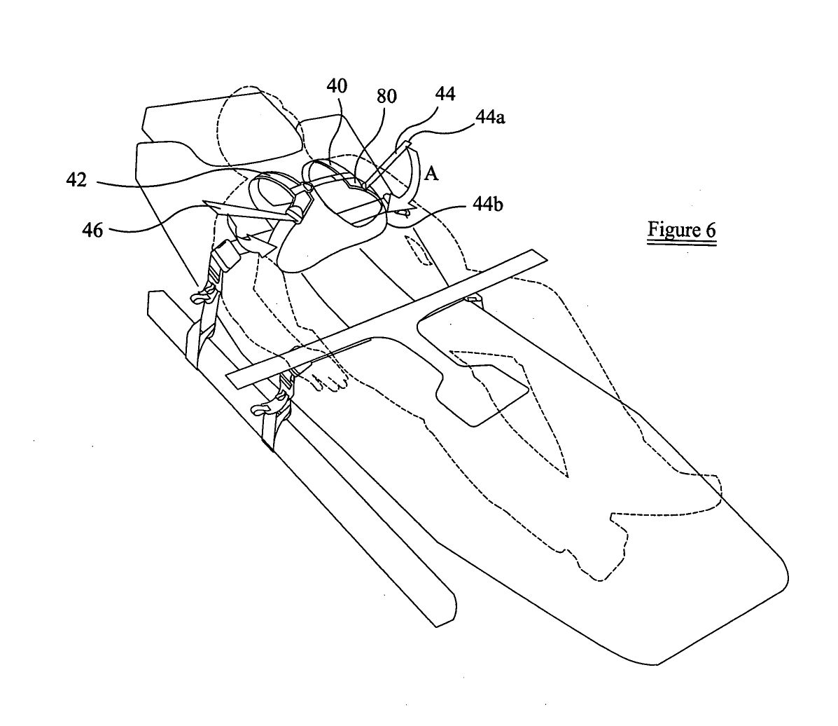

As shown in FIG. 6, the first underarm strap 44 is passed through an eye 80 attached to the first shoulder strap 40. An outer portion 44a of the first underarm strap 44 is then bent back in the direction of arrow A to engage an inner portion 44b of the first underarm strap 44. Attached to the outer portion 44a is one half of a hook and loop fastener which attaches to the other half of the hook and loop fastener attached to the inner portion 44b, securing to the underarm strap 44 to the first shoulder strap 44.

The second underarm strap 46 is secured to the second shoulder strap 42 in the same way.

It will be noted that, once restrained, the patient's chest is exposed, which is desirable from the point of view of the clinician as exposure of the chest facilitates clinical intervention.

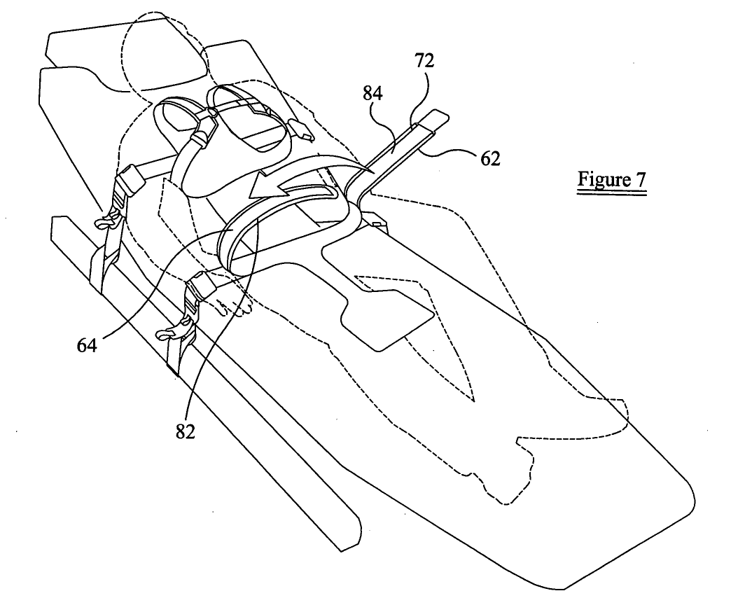

Referring to FIG. 7, the second waist strap 64 is passed over across the patient's abdomen. In this position, the exposed surface 82 comprises one half of a hook and loop fastener. The first waist strap 62 is passed over the top of the second waist strap 64, in the direction of the arrow, such that a leading surface 84 of the waist strap second element 72 engages the second waist strap surface 82. The waist strap second element leading surface 84 of the comprises the other half of the hook and loop fastener, the two halves coming together to secure the first waist strap 62 to the second waist strap 64.

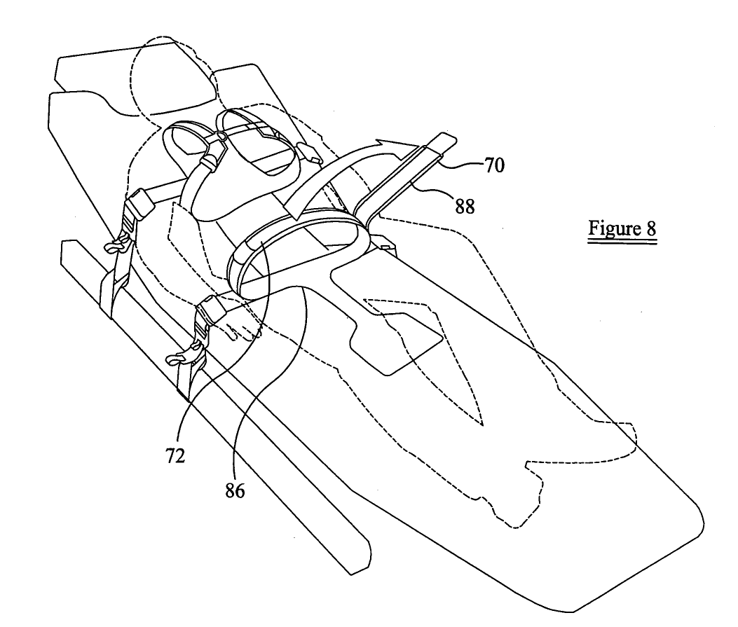

Referring now to FIG. 8, the waist strap first element 70 is peeled away from the waist strap second element 72 to expose a pair of surfaces 86,88. Attached to the waist strap exposed surfaces 86,88 are both halves of a hook and loop fastener, such that the first and second elements 70,72 can be attached to each other.

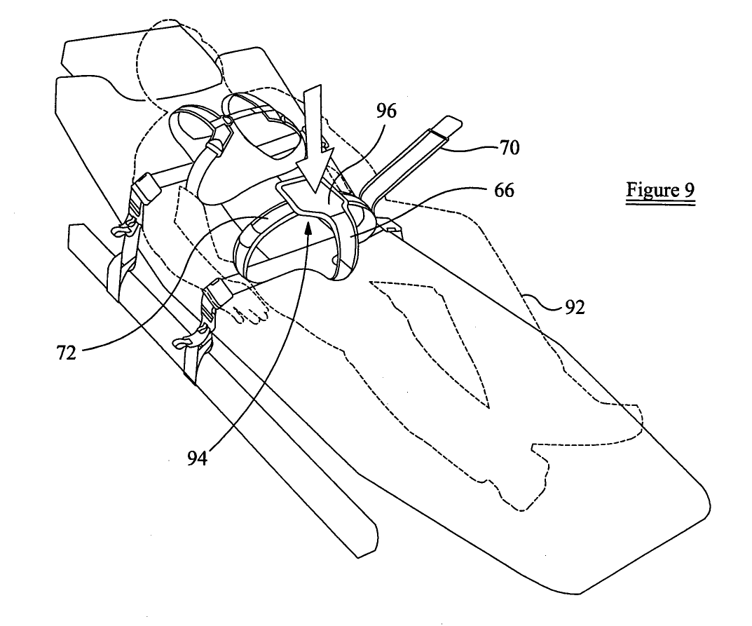

Referring to FIG. 9, the crotch strap 66 is folded up between the patient's legs 92. A front side 94 and a back side 96 of the crotch strap 66 also comprise both halves of a hook and loop fastener and are arranged such the crotch strap front side 94 engages and fastens to the waist strap second element 72. The waist strap first element 70 is then folded back over the top of, and attaches to, the crotch strap 66, the hook and loop fastener halves engaging and securing the waist straps 62, 64 and the crotch strap 66.

The patient 12 is now restrained in the position shown in FIG. 1.

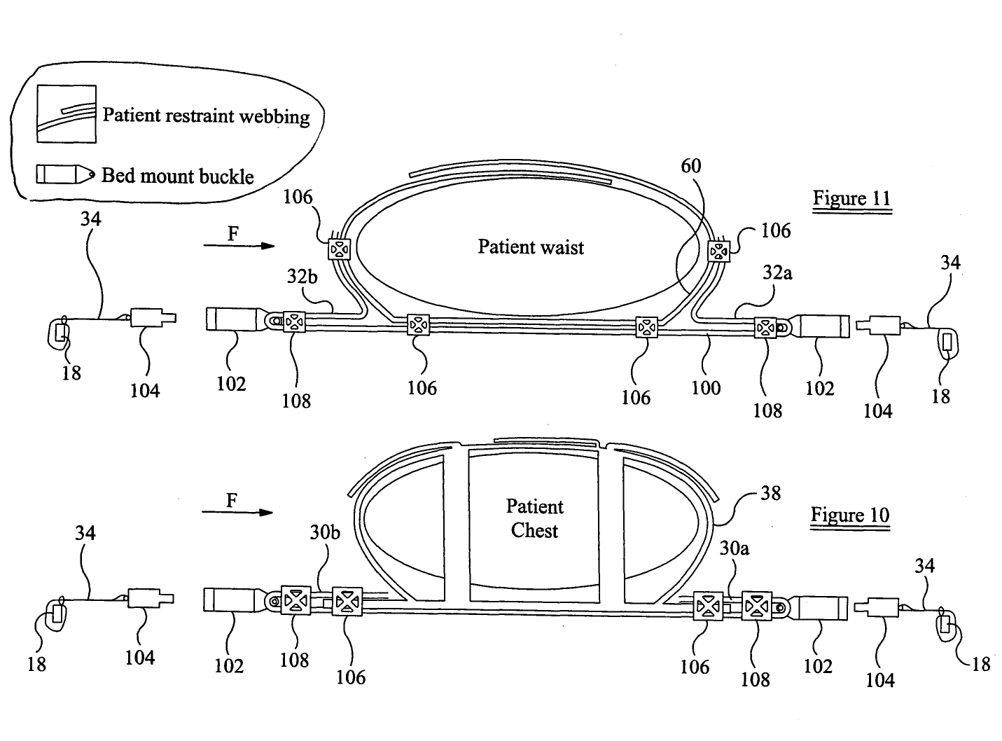

The attachment devices 24 will now be discussed with reference to FIGS. 10 and 11.

FIG. 10 shows a schematic view of the upper torso attachment straps 30 and the upper torso restraint 20. As can be seen from FIG. 10, the attachment straps 30a,30b are fashioned from a single piece of webbing 100. The attachment straps 30a,30b include female buckle portions 102 which engage male buckle portions 104 attached to the frame straps 34.

The attachment straps 30 are secured to the upper torso back portion 38 by stitchings 106. The multiple stitchings 106 are provided to prevent the attachment straps 30 from peeling off the upper torso back portion 38, which can occur in the event of sudden force being applied in the direction of arrow F.

Further stitchings 108 secure the female buckle portions 102 with respect to the attachment straps 30a,30b.

FIG. 11 shows a schematic view of the lower torso attachment straps 32 and the lower torso restraint 22. The arrangement of the straps 32 of FIG. 11 is similar to that of FIG. 10 and is not repeated for brevity.

In both FIGS. 10 and 11, the frame straps 34 are wrapped around the frame 18 and pulled tight.

Various modifications and improvements may be made to the described embodiment without departing from the scope of the invention. For example, although shown in connection with a framed stretcher, the restraint harness 10 is suitable for use with other types of support apparatus, such as a spinal board. If no frame is present, a male buckle could be provided to one attachment strap for engaging with the female buckle of the other attachment strap. In this way the attachment straps can be passed around the back of the support device and connected together to grip the support device.

Furthermore, the frame straps could be lengthened to permit the patient to be restrained in the recovery position. In this position a support, such as a rolled up towel, could be placed down the length of the patient's back to provide support for the patient.

1-74. (canceled)

75. A restraint harness for a child, the restraint harness comprising:
an upper torso restraint;
a lower torso restraint; and
at least one attachment device for attaching the upper and lower torso restraints to a stretcher or other device;
wherein, in use, the upper torso restraint and the lower torso restraint restrain independently of each other.

76. The restraint harness of claim 75 wherein the restraint harness comprises straps which extend in at least two directions over the user's body, the restraint harness comprising at least one over shoulder strap a plurality of side straps and at least one crotch strap wherein each strap comprises a first end and a second end, the first end of each strap being fixedly connected to the upper torso back portion.

77. The restraint harness of claim 75, wherein the upper torso restraint is at least a three point restraint.

78. The restraint harness of claim 75, wherein the upper torso restraint is a four point restraint.

79. The restraint harness of claim 76, wherein the upper torso restraint comprises an upper torso back portion, first and second shoulder strap and first and second underarm straps, the shoulder straps adapted to pass over the user's shoulders to the user's chest and the underarm straps adapted to pass up the user's sides to the user's chest.

80. The restraint harness of claim 79, wherein each of the underarm straps is releasably connectable to at least one of the shoulder straps or vice versa.

81. The restraint harness of claim 79, wherein the underarm straps are releasably connectable to each other.

82. The restraint harness of claim 79, wherein the shoulder straps are releasably connectable to each other by means of a neck portion, the neck portion comprising a neck strap.

83. The restraint harness of claim 79, wherein the upper torso back portion and shoulder straps define an aperture adapted to be passed over a user's head.

84. The restraint harness of claim 77, wherein the lower torso restraint is a three point restraint, the lower torso restraint comprising a lower torso back portion, first and second waist straps and a crotch strap.

85. The restraint harness of claim 84, wherein each strap comprises a first end and a second end, the first end of each strap being fixedly connected to the lower torso back portion.

86. The restraint harness of claim 84, wherein the waist straps pass over the user's hips to the user's abdomen, and the crotch strap passes between the user's legs to the user's abdomen, the crotch strap being adapted to be sandwiched between the waist straps.

87. The restraint harness of claim 84, wherein the waist straps are connectable to each other, the first waist strap comprising a securing device for securing the second waist strap and the crotch strap, the securing device comprising first and second elements, the crotch strap being adapted to be received between the first and second elements.

88. The restraint harness of claim 87, wherein the first and second elements have opposing surfaces adapted to be received the crotch strap therebetween, the crotch strap having an upper surface and lower surface, each of the upper and lower surfaces adapted to connectably engage with one of the opposing surfaces of the securing device elements.

89. The restraint harness of claim 75, wherein the upper torso restraint and the lower torso restraint are connected.

90. The restraint harness of claim 89, wherein the upper torso restraint and the lower torso restraint are movable connected.

91. The restraint harness of claim 89, wherein the restraint harness further comprises at least one connection element to connect the upper torso restraint and the lower torso restraint.

92. The restraint harness of claim 91, wherein the/each connection element fixedly attaches the upper and lower torso restraints.

93. The restraint harness of claim 91, wherein the/each connection element can elastically deform.

94. The restraint harness of claim 75, wherein the restraint harness is movable between a useable configuration and a stored configuration.

95. The restraint harness of claim 94, wherein the restraint harness comprises an elastically deformable storage device for storing the restraint harness when not in use, the storage device being integral with the restraint harness and adapted to compress the restrain harness when in the stored configuration.

96. The restraint harness of claim 75, wherein the restraint harness further comprises a plurality of attachment straps fixedly connected to one or both of the upper and lower torso back portions.

97. The restraint harness of claim 96, wherein the/each attachment strap comprises a first portion and a second portion, each attachment strap first portion is connected to one of the torso back portions and each attachment strap second portion is adapted to be connected to a frame of a bed or stretcher or other similar device.

98. The restraint harness of claim 97, wherein the attachment straps first and second portions are releasably attachable to each other by means of a buckle.

99. The restraint harness of claim 75, wherein the restraint harness is adapted to permit a patient to be restrained on the user's back or the user's side.

100. The restraint harness of claim 1, wherein the upper and lower torso restraints portions and/or the/each connection element comprise a wipe clean material and at least one padded portion.

101. A restraint harness comprising;
an upper torso restraint;
a lower torso restraint; and
at least one attachment device for attaching the upper and lower torso restraints to a stretcher or other device;
wherein, in use, the upper torso restraint and the lower torso restraint restrain a patient whilst leaving the patient's chest substantially uncovered.
